(19) United States
(12) Patent Application Publication
(10) Pub. No.: US 2009/0013769 A1
(43) Pub. Date: Jan. 15, 2009

(54) ASSAY FOR OILS

(76) Inventors: Tony Larson, York (GB); Ian Alexander Graham, York (GB)

Correspondence Address: KENING LI;PINSENT MASONS, c/o BAKER DONELSON BEARMAN CALDWELL& BERKOWITZ, PC, 555 ELEVENTH STREET, NW, SIXTH FLOOR, WASHINGTON, DC, 20004, US

(21) Appl. No.: 11/573,860

(22) PCT Filed: Aug. 15, 2005
(86) PCT No.: PCT/GB2005/003193

§ 371 Date: Jun. 14, 2007

(30) Foreign Application Priority Data
Aug. 19, 2004 (GB) 0418529.4

(51) Int. Cl. G01N 33/03; G01N 30/60; G01N 30/88

(52) U.S. Cl. 73/61.53; 210/656

(57) ABSTRACT

We describe a method for the detection of triacylglycerols (TAGs) in a biological sample.

### FIELD OF THE INVENTION

The invention relates to a method for the detection of triacylglycerols (TAGs) in a biological sample.

### BACKGROUND OF THE INVENTION

Commercially important fats and oils of animal and plant origin consist primarily of simple lipids referred to as triacylglycerols (TAGs). These include all vegetable oils, (e.g. rapeseed, maize, olive, palm, sunflower, evening primrose) and animal fats such as tallow, lard, and dairy products such as butter, milk and cheese. Many synthetic or semi-synthetic products such as margarine also include TAGs. TAGs are also a main starter material for the manufacture of pharmaceutical, cosmetic and nutraceutical products.

A TAG consists of a trihydric alcohol glycerol esterified with long chain fatty acids. The major biosynthetic pathway for TAGs in both plant and animal tissues is the α glycerophosphate or sn-glycerol phosphate pathway. In this pathway sn glycerol-3-phosphate produced by the enzyme glycerol kinase on free glycerol is acylated sequentially by specific acyl transferases at positions sn-1 and sn-2 to form phosphatidic acid. The phosphate group is removed by phosphatidate phosphatase and the resultant diacylglycerol is acylated by a further acyl transferase to form a triacyl-sn-glycerol (at position sn-3). Naturally occurring TAGs therefore exist in enantiomeric forms with each position of the sn-glycerol moiety esterified with different or the same fatty acids (i.e. fatty acids are esterified at positions sn-1, sn-2 and sn-3). TAG composition can therefore vary considerably depending on fatty acid content.

For example, seed TAGs varies due to plant species, plant variety within a species, geographic location and environmental conditions. Seed TAG composition may be further altered by post-harvest processing, long-term storage and deliberate blending/adulteration. The analysis of TAG composition can be used to determine the origin and purity of seed oils. It is also important to analyse TAG content because the various properties of TAGs is to a large extent determined by the fatty sn isomers. TAG analysis traditionally involves the measurement of total TAGs as glycerol equivalents. This gives an indication of oil yield, but cannot impart any information about oil quality or purity.

Methods for determining TAG species present in oil have been developed to monitor oil quality and purity. These include normal-phase high performance liquid chromatography (HPLC). For example, ES2177375 describes the use of HPLC in the detection of TAGs in vegetable oils, in particular olive oil. A further example is described in WO92/12421 that describes the analysis of mixtures of TAGs by mass spectroscopy. More recently non-aqueous reverse-phase HPLC (NARP) methods using C18 columns have been used in the analysis of TAGs (Jandera et al. 2004, J. Chromatogr. A 1030:33-41; Holcapek et al. 2003, J. Chromatogr. A 1010:195-215). NARP methodology optimises the use of C18 columns for the analysis of highly lipophilic compounds such as TAGs, resulting in rapid equilibration times between runs and more reproducible separations compared to traditional normal-phase HPLC methods. NARP methods are therefore particularly suited to complex oil samples where many TAG species may be present.

A problem associated with existing methods to analyse oil samples for TAG content is that analyses require very long run times using normal phase or NARP HPLC. There is a need for faster analytical methods to enable a more rapid testing of oils to determine their TAG content. We describe a method that greatly increases the speed of analysis of oil samples for TAG content.

### DESCRIPTION OF THE INVENTION

According to an aspect of the invention there is provided a method for the analysis of a composition comprising triacylglycerol comprising the steps of:
i) providing a preparation comprising triacylglycerol;
ii) applying the preparation in (i) to a column wherein said column comprises a separation material of a lipophilic polymer; and
iii) separating the triacylglycerol content of said preparation.

In a preferred method of the invention said composition is derived from a plant. Preferably said preparation is derived from the seed of a plant.

In a further preferred method of the invention said plant is an oil seed plant.

Preferably said oil seed plant is selected from the group consisting of: cotton, soybean, safflower, sunflower, cocoa, Brassica spp, maize, alfalfa, palm, coconut, rapeseed, olive, evening primrose, linseed, peanut, hemp, borage, calendula, camelina, crambe, echium, lesquerella, castor, limnanthes, lunaria, and avocado.

In a preferred method of the invention said oil seed plant is olive.

In an alternative preferred method of the invention said composition is derived from an animal. Preferably said composition comprises an edible animal fat.

In a preferred method of the invention said animal is a cow.

In a preferred method of the invention said composition is a diary product. Preferably said dairy product is selected from the group consisting of milk, butter, and cheese. Preferably said dairy product is an infant milk formula.

In a further preferred method of the invention said column is a high performance liquid chromatography column (HPLC).

HPLC is a very well known method for the separation and analysis of solute molecules. The separation process is effected through liquid chromatography and relies on the fact that a number of component solute molecules in a sample stream of fluid (mobile phase) flowing through a packed bed of particles (stationary phase) can be efficiently separated from one another with a high degree of resolution. This is based on the fact that individual components in the mobile phase have a different affinity for the stationary phase and therefore a different rate of migration and exit through the column. The efficiency of separation is determined by the amount of spreading through the column which is determined by the column composition. We have found that a column that has a very high lipophilic content is surprisingly effective with respect to the separation of triacylglycerol from a complex mixture.

In a preferred method of the invention said column comprises a highly lipophilic polymer.

“Highly lipophilic” refers to a separation column that is effective at retaining non-polar compounds such as long-chain hydrocarbons and related structures.

“Polymeric” refers to the structure of the base material used to manufacture the packing of the column, for example a polymer based on styrene-benzene, although other column packing materials can be used in the method of the invention, for example silica-based packings.

In a further preferred method of the invention said polymeric material is non-encapped.

“Non-endcapped” refers to the treatment of the polymeric packing material during manufacture. The polymeric packing material in an “end-capped” column are treated with a blocking compound in the final stages of manufacturer to bind any unwanted active sites that would otherwise affect the separation characteristics of the column. Polymeric columns do not typically have these active sites (unlike, for example, silica-based columns, where the packings have residual silanol groups), so they are not end-capped.

In a further preferred embodiment of the invention said lipophilic polymer comprises hydrocarbon chains that confer lipophilicity.

In a preferred method of the invention said hydrocarbon chains comprise at least 19 carbon atoms.

In a further preferred method of the invention said hydrocarbon chains comprise at least 20, 21, 22, 23, 24, 25, 26, 27, 28, or 29 carbon atoms.

In a preferred embodiment of the invention said hydrocarbon chain comprises at least 30 carbon atoms. Preferably said hydrocarbon chain consists of 30 carbon atoms.

In a further preferred method of the invention said method further comprises the steps of:
i) detecting and collating the triacylglycerol content of said preparation;
ii) collating the data into a data analysable form; and optionally
iii) providing an output for the analysed data.

According to a further aspect of the invention there is provided the use of a high performance liquid chromatography column comprising a lipophilic polymeric material for the separation of triacylglycerols.

In a preferred embodiment of the invention said column comprises hydrocarbon chains comprising at least 19 carbon atoms.

In a further preferred embodiment of the invention said column comprises hydrocarbon chains comprising at least 20, 21, 22, 23, 24, 25, 26, 27, 28, or 29 carbon atoms.

In a preferred embodiment of the invention said column comprises hydrocarbon chains comprising at least 30 carbon atoms. Preferably said column comprises hydrocarbon chain consisting of 30 carbon atoms.

### BRIEF DESCRIPTION OF THE DRAWINGS

The invention will now be described by example only and with reference to the following table and Figures:

Table 1 illustrates source olive oils used for method evaluation;

FIG. 1 illustrates fatty acid profile determined by GC-FID analysis of fatty acid methyl esters (FAMEs) from transmethylated oil samples;

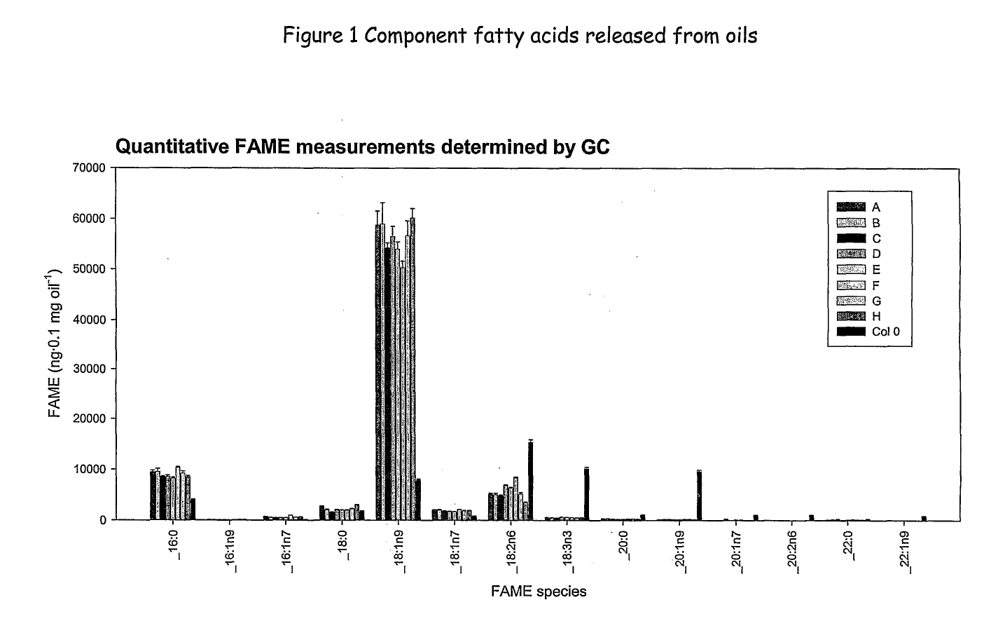

FIG. 2 illustrates PCA (principal components analysis) using quantitative GC FAME data (14 variables);

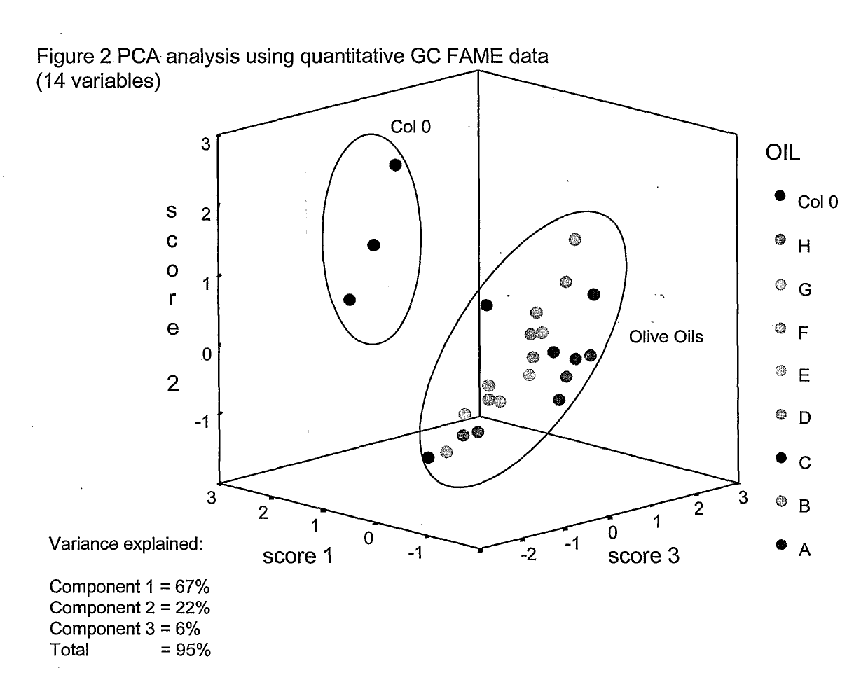

FIG. 3 illustrates an analysis of olive oil A using 2 carbon 18 (C18) columns for separation;

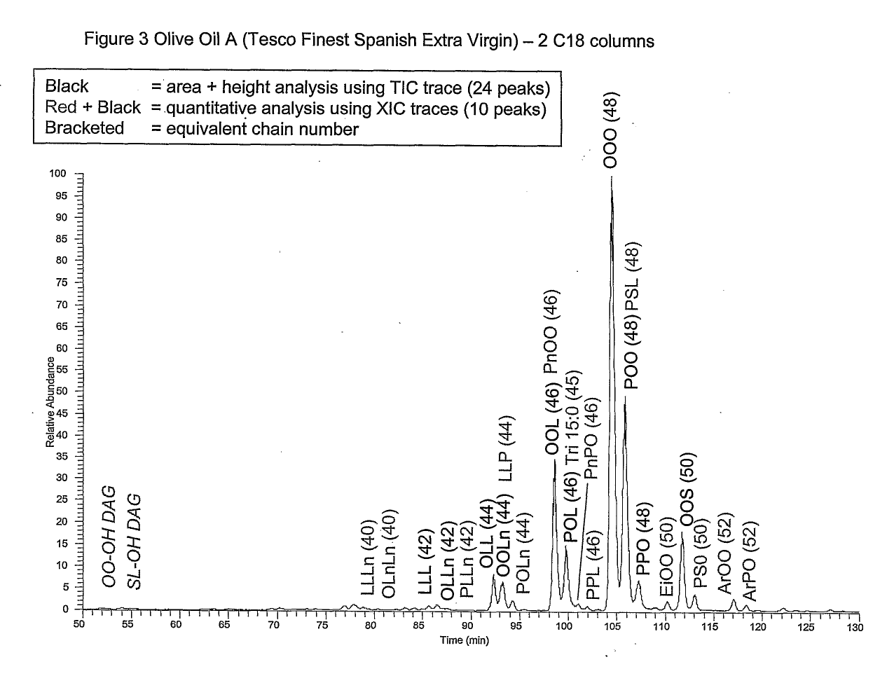

FIG. 4 illustrates an analysis of olive oil A using a carbon 30 (C30) column for separation;

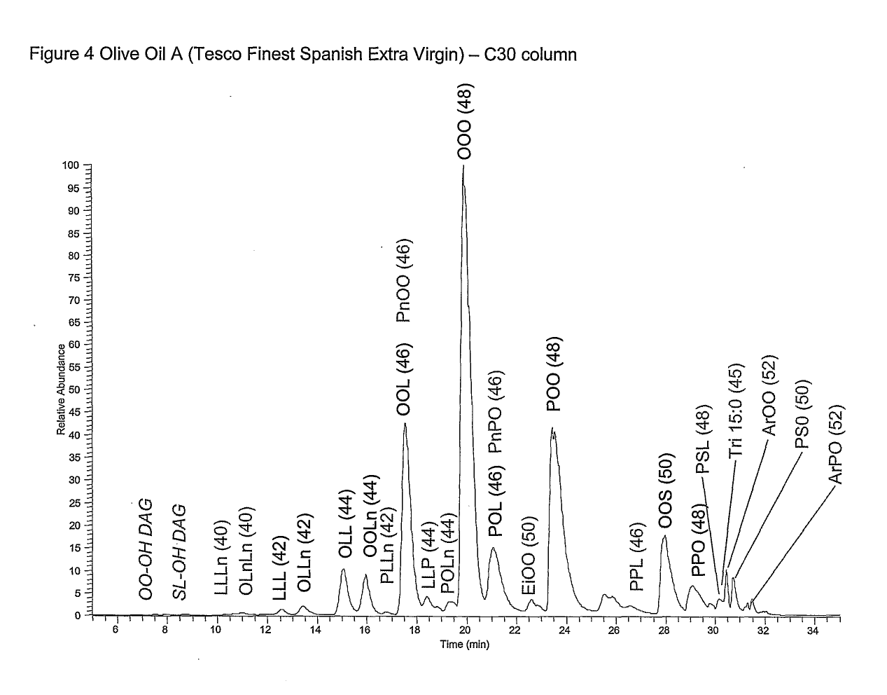

FIG. 5 illustrates TAG standardised amount analysis using a C30 column;

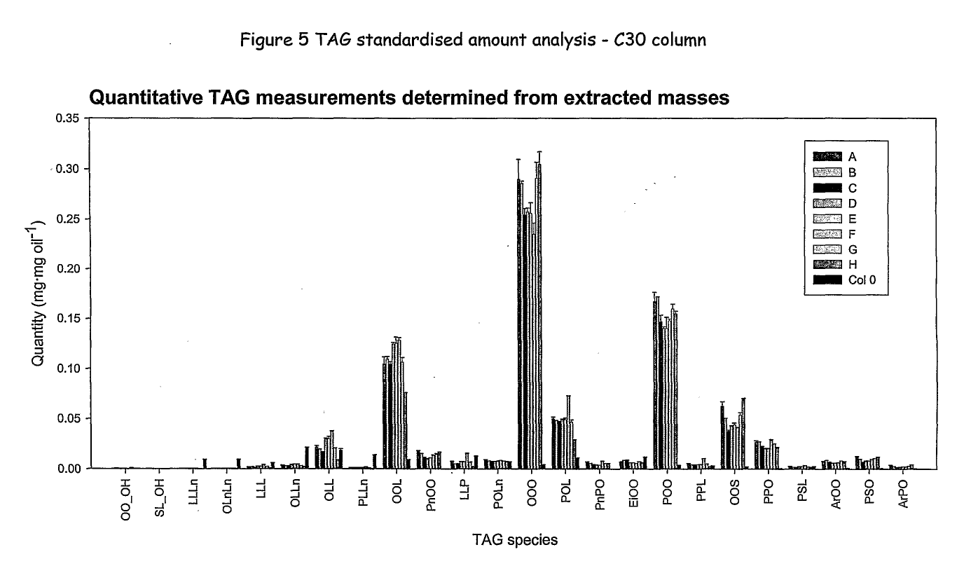

FIG. 6 illustrates PCA analysis using quantitative extracted ion data for TAGs using 2 C18 columns for separation (24 variables);

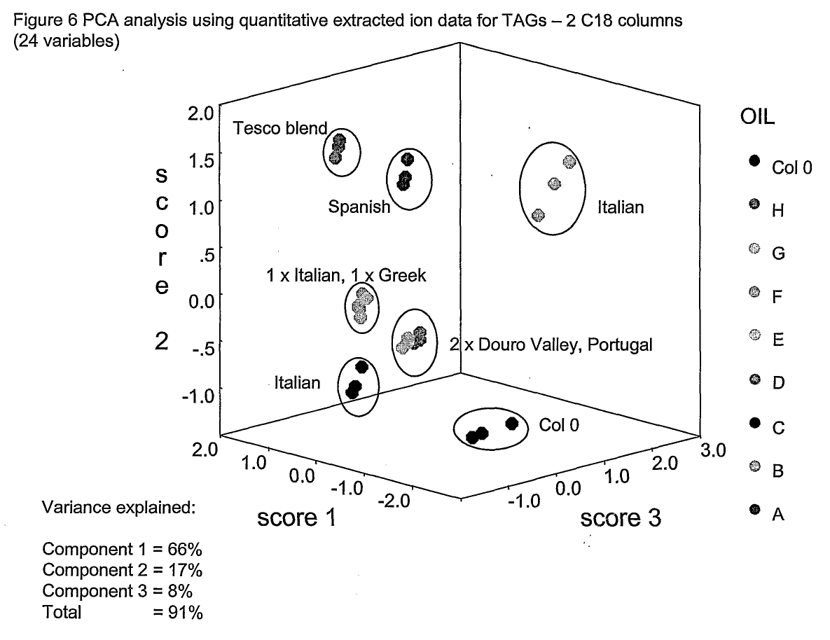

FIG. 7 illustrates PCA analysis using quantitative extracted ion data for TAGs using a C30 column for separation (24 variables);

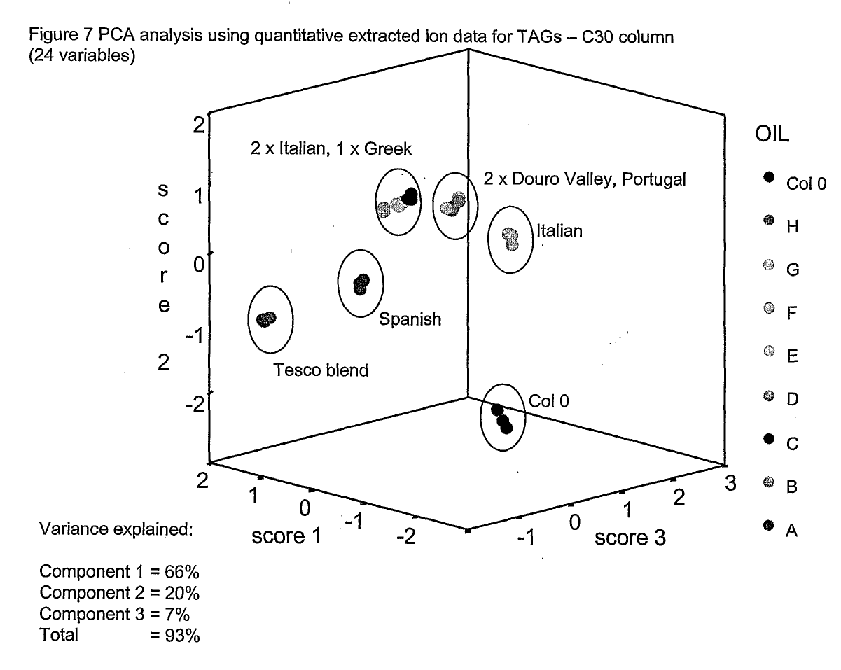

FIG. 8 illustrates TAG area amount analysis using a C30 column for separation; and

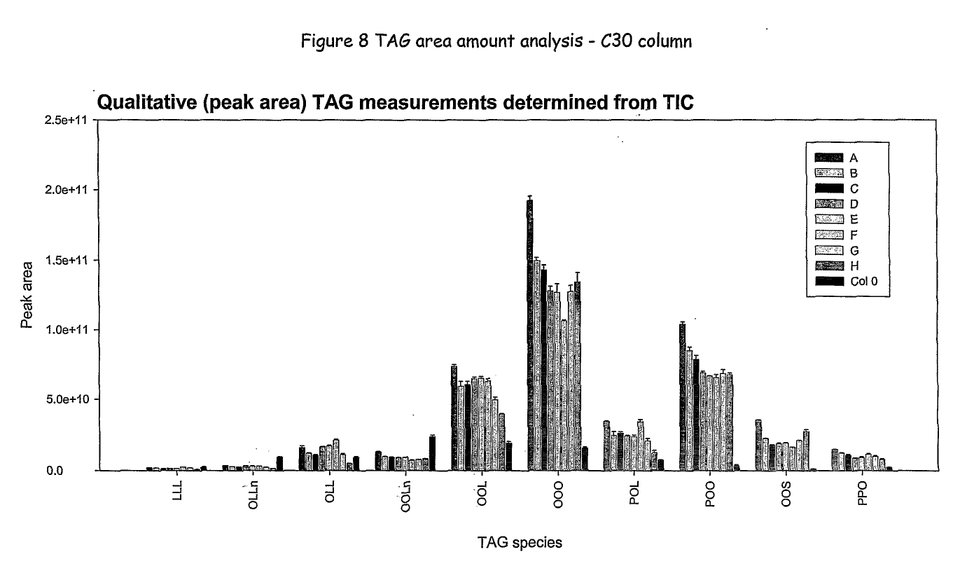

FIG. 9 illustrates PCA analysis using semi-quantitative total ion current area data for TAGs using a C30 column for separation (10 variables).

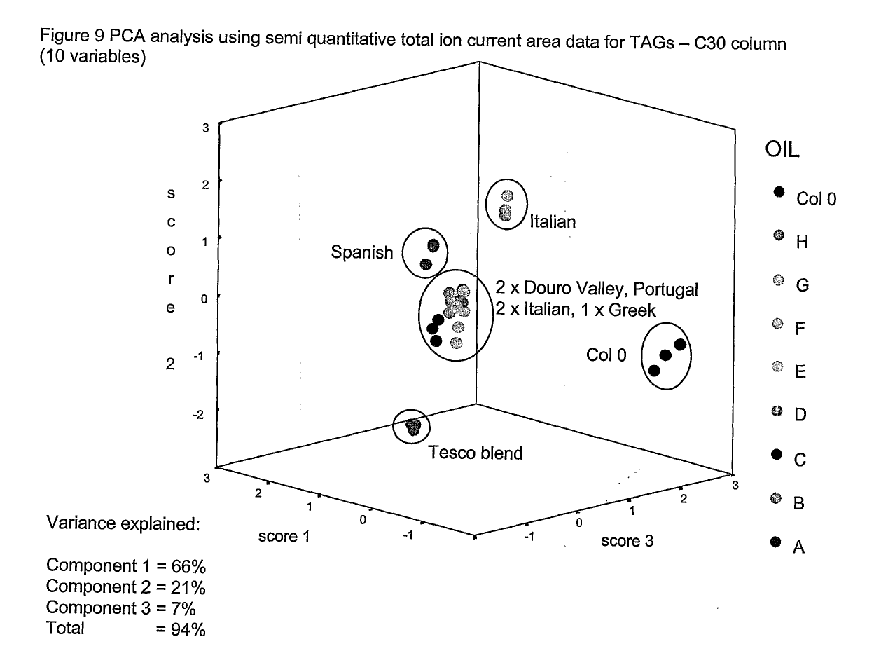

### EXAMPLES

Materials and Methods

Sample Preparation

“Extra Virgin” branded olive oils were purchased from the supermarket the day before they were brought into the laboratory. Oils were selected based on their stated origin; variety information is unknown. The bottles were opened and the oils diluted, aliquoted, and stored at −20° C. before analysis. 100 mg·mL-1 (w/v) oil stocks were made up in tert-methyl butyl ether (MTBE). From these, 10 mg·mL-1 dilutions were made up containing 0.05 mg·mL-1 tripentadecanoin as an internal standard.

LC Conditions (Method Under Evaluation)

Instrumentation: Thermo Separations Products SCM1000 degasser; P4000 gradient pump; AS3000 autosampler maintained at 4° C., fitted with a 100 μL stainless steel loop and operated in pull-loop mode . Mobile Phases: A=60% ethanol 40% acetonitrile 0.2% formic acid (v/v); B=tetrahydrofuran 0.2% formic acid. Gradient Profile: 0-5 min 100% A; 5-25 min to 70% A 30% B; 25-30 min to 100% B; 30-35 min 100% B; 35-35.1 min to 100% A; 35.1-40 min 100% A; 1 mL·min-l. 1 min equilibration time between runs. Column: YMC-Pack YMC C30, 250×4.6 mm, part number CT99S053546; maintained at 30° C. Injection conditions: 10 μL injection volume; needle rinse solvent=methanol.

LC Conditions (Existing Method—Holcapek et al. (2003) J. Chromatography A 1010:195-215)

Instrumentation: As above. Mobile Phases: A=Water; B=acetonitrile; C=2-propanol. Gradient Profile: 0 min 30% A 70% B; 0-20 min to 100% B; 20-36 min 100% B; 36-132 min to 40% B 60% C; 132-135 min to 30% a 70% B; 135-40 min 30% A 70% B. 1 min equilibration time between runs. Column: Two Waters Nova-Pak 100×3.9 mm C18 columns connected in series, part number WAT086344; maintained at 40° C. Injection conditions: As above.

Mass Spectroscopy Conditions

Instrumentation: Thermo LCQ ion trap mass spectrometer. Source=atmospheric pressure chemical ionization; positive ionization mode; vaporizer temperature 500° C.; N2 sheath flow 60 units; N2 aux flow 60 units; corona discharge current 5 μA; capillary temperature 150° C.; capillary voltage 15 V.

Data collection: LC flow diverted to MS 5-35 min; full scan MS data 500-1500 m/z with automatic gain control on; data dependent fragmentation at normalised collision energy of 35% to identify TAG fatty acid components. Peak integration: total ion current or extracted ions corresponding to TAG ammonium adducts used for peak integration using the ICIS algorithm in the software package Xcalibur 1.2 (Thermo).

Data Analysis

Triplicate samples were injected from separate vials. TAG amounts were calculated as extracted ion peak area relative to internal standard peak area for all peaks, or absolute peak area for non-co eluting peaks detected in total ion current mode. Peak tables were exported from Xcalibur for principal components analysis using SPSS 11.0 software, using a correlation matrix and varimax with kaiser normalization rotation.

### RESULTS

The results indicate that simple fatty acid methyl ester profiling is unable to distinguish the origin of different olive oil samples. However, TAG measurements do enable the olive oils to be separated according to their origin by PCA analysis. This is not surprising, as TAGs are produced in all oilseed species by several acyltransferase reactions in addition to the fatty acid synthesis steps. TAG composition reflects the multivariate influences of plant species, growth, harvest and processing conditions on the summed biochemical steps leading to seed oil accumulation. Therefore, TAG analysis is powerful tool that has the potential to identify plant oil geographical origins, adulteration, and oil degradation due to post-harvest processing and storage.

Despite the potential of TAG analysis to distinguish between plant oils, commercial application is limited by the long HPLC run times needed to resolve peaks representing individual compounds from extracted samples. Analysis times between one and two hours are typical for normal-phase and NARP HPLC methods, which precludes the use of these methods for rapid, high-throughput screening. Shorter analysis times can be used if coeluting peaks are resolved by mass spectrometry. However, this relies on the coeluting compounds having unique masses that is not always the case, especially for complex oils. In addition, it is desirable to maximise the chromatographic resolution to allow the use of more cost-effective and robust analog detectors, such as ultraviolet (UV) or evaporative light scattering detectors (ELSD), for routine screening.

In order to maximise chromatographic resolution and minimise run time, we employed a C30 column for NARP HPLC separation of TAGs. This allowed a four-fold decrease in analysis time compared to using an established NARP HPLC protocol using C18 columns. Although the analysis time was decreased, chromatographic resolution was retained because of the unique selectivity of the C30 column. PCA analysis was successful in separating the different olive oils using either extracted mass data (which included data from coeluting peaks) or total ion current data (fewer variables; data only from chromatographically resolved peaks) from analyses with the C30 column. This demonstrates that the C30 column could be used successfully to differentiate olive oil samples using mass spectrometric, UV, or ELSD detectors in less than 35 minutes per sample.

TABLE 1
Olive Oil Samples
All purchased from Tesco Supermarket, Seacroft, Leeds 22 Apr. 2004

| | | | | | | Stated Fat types, g/100 g | | |
|---|---|---|---|---|---|---|---|---|
| | | | | | | (measured; all SD < 5%) | | |
| | | | | | | | Mono- | Poly- |
| ID | Name | Bottle size (mL) | Produced in | Packed in | Best before end | Saturates | unsaturates | unsaturates |
| A | Tesco finest Spanish | 500 | Spain | Cheshunt UK | February 2005 | 14.3 (15.6) | 73 (77.3) | 8.2 (7.1) |
| | Extra Virgin Olive Oil | | | | | | | |
| C | Tesco finest Tuscan | 500 | Tuscany, Italy | Cheshunt UK | October 2004 | 14.3 (14.4) | 73 (78.1) | 8.2 (7.4) |
| | Extra Virgin Olive Oil | | | | | | | |
| D | Almendra Estate Extra | 500 | Douro Valley, Portugal | Portugal | December 2005 | NA (14.1) | NA (76.3) | NA (9.6) |
| | Virgin Olive Oil | | | | | | | |
| F | Filippo Berlo Extra | 250 | Italy | Lucca, Italy | August 2005 | 14.5 (17.0) | 63.5 (71.2) | 9.3 (11.8) |
| | Virgin Olive Oil | | | | | | | |

1. A method for the analysis of a composition comprising triacylglycerol comprising the steps of:
i) providing a preparation comprising triacylglycerol;
ii) applying the preparation in (i) to a column wherein said column comprises a separation material of a lipophilic polymer; and
iii) separating the triacylglycerol content of said preparation.

2. A method according to claim 1 wherein said composition is derived from a plant.

3. A method according to claim 2 wherein said preparation is derived from the seed of a plant.

4. A method according to claim 2 wherein said plant is an oil seed plant.

5. A method according to claim 4 wherein said oil seed plant is selected from the group consisting of: cotton, soybean, safflower, sunflower, cocoa, Brassica spp, maize, alfalfa, palm, coconut, rapeseed, olive, evening primrose, linseed, peanut, and avocado.

6. (canceled)

7. A method according to claim 1 wherein said composition is derived from an animal.

8. A method according to claim 7 wherein said composition comprises an edible animal fat.

9. A method according to claim 7 wherein said animal is a cow.

10. A method according to claim 7 wherein said composition is a diary product.

11. (canceled)

12. A method according to claim 1 wherein said preparation is an infant milk formula.

13. A method according to claim 1 wherein said column is a high performance liquid chromatography column.

14. A method according to claim 13 wherein said column comprises a highly lipophilic polymer.

15. A method according to claim 13 wherein said polymeric material is non-encapped.

16. A method according to claim 14 wherein said lipophilic polymer comprises hydrocarbon chains that confer lipophilicity.

17. A method according to claim 16 wherein said hydrocarbon chains comprise at least 19 carbon atoms.

18. A method according to claim 16 wherein said hydrocarbon chains comprise at least 20, 21, 22, 23, 24, 25, 26, 27, 28, or 29 carbon atoms.

19. A method according to claim 16 wherein said hydrocarbon chain comprises at least 30 carbon atoms.

20. A method according to claim 16 wherein said hydrocarbon chain consists of 30 carbon atoms.

21. A method according to claim 20, wherein said method further comprises the steps of:
i) detecting and collating the triacylglycerol content of said preparation; and
ii) collating the data into a data analysable form; and optionally

22-26. (canceled)

27. The method according to claim 21, further comprising:
iii) providing an output for the analysed data.

28. The method according to claim 1, wherein the triacylglycerol content is separated via high performance liquid chromatography.
